(19) United States
(12) Patent Application Publication
(10) Pub. No.: US 2005/0272130 A1
(43) Pub. Date: Dec. 8, 2005

(54) Methods and compositions relating to prion-only transmission of yeast strains

(76) Inventors: Chin-Yen King, Redmond, WA (US); Ruben Diaz-Avalos, Tallahassee, FL (US)

Correspondence Address: NEEDLE & ROSENBERG, P.C., SUITE 1000, 999 PEACHTREE STREET, ATLANTA, GA, 30309-3915, US

(21) Appl. No.: 11/083,055

(22) Filed: Mar. 17, 2005

Related U.S. Application Data
(60) Provisional application No. 60/553,816, filed on Mar. 17, 2004.

(51) Int. Cl. C12N 15/09; G01N 33/53; C07H 21/04; C07K 14/47

(52) U.S. Cl. 435/69.3; 435/320.1; 435/325; 530/350; 536/23.5

(57) ABSTRACT

Disclosed are compositions and methods related to prion-only transmission of yeast strains.

### I. CROSS REFERENCE TO RELATED APPLICATIONS

This application claims priority to provisional patent application number 60/553,816, filed Mar. 17, 2005, which application is hereby incorporated by this reference in its entirety.

### II. BACKGROUND

A prion is a small proteinaceous infectious particle which resists inactivation by procedures that modify nucleic acids. The possibility that proteins alone may transmit an infectious disease is currently being debated.

Prion diseases are often called spongiform encephalopathies because of the post mortem appearance of the brain with large vacuoles in the cortex and cerebellum. Probably most mammalian species develop these diseases. Specific examples include Scrapie (sheep), TME (transmissible mink encephalopathy), CWD (chronic wasting disease found in muledeer and elk), BSE (bovine spongiform encephalopathy, found in cows), CJD (Creutzfeld-Jacob Disease in humans) GSS (Gerstmann-Straussler-Scheinker syndrome in humans), FFI (Fatal familial Insomnia), Kuru, and Alpers Syndrome.

Ingested prions may be absorbed across the gut wall at Peyers patches. These are a part of the MALT, or mucosal associated lymphoid tissue. MALT presents microorganisms to the immune system in a contained and ideal fashion, facilitating a protective immune response. Prions can be taken up in the same way. Lymphoid cells then phagocytose the particle and travel to other lymphoid sites such as nodes, the spleen and tonsils. The prion can replicate at these sites. Many of these sites are innervated and eventually the prion gains access to a nerve and then propagates back up the axon to the spinal cord and eventually to the brain. SCID mice are resistant to a prion challenge, confirming the importance of the lymphoid system. Furthermore, PrP (prion protein) null mice i.e., those in which both alleles have been disrupted, PrP^{o/o}, cannot be infected. PrP^{o/o} mice carrying a PrP^{+/+} brain graft can develop pathology following an intracerebral injection but only in the graft. PrP^{o/o} mice+graft+a reconstituted PrP^{+/+} lymphoreticular system are resistant to a prion challenge. This indicates another compartment in addition to the brain and the LRS must express PrP, if a peripheral prion challenge is to be successful. That compartment is probably a nerve. Mature B lymphocytes are also now known to be required for the development of the disease following infection from a peripheral route.

Tg PrP mice and knockout mice have been informative in prion research. In a further series of experiments PrP^{o/o} mice have had a hamster PrP transgene incorporated. This transgene has been put under the transcriptional control of either the glial fibrillary acidic protein promoter, or the neuron specific enolase promoter such that hamster PrP is only expressed in either the glial cells or neuronal cells. Following an intracerebral prion challenge both groups of animals can replicate the prion agent and develop disease pathology. So either glial cells or neural cells can propagate the disease independently. The fact that PrPsc (for scrapie) intracerebral injection alone in PrP^{o/o} mice, does not cause pathology means that cells must be making PrP for a pathological result. It is known that cytokine levels are elevated in the later stages of a prion infection. Astrocytes and other glial cells produce these and presumably have a key role in pathogenesis. This conclusion is supported by the fact that murine retroviruses are known which infect glial cells and result in a spongiform degeneration of the brain.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows proof of strain-specific infectivity of [PSI] prion aggregates. a, Experimental scheme to demonstrate that Sup35 amyloid particles transmit the [PSI] strain phenotype. Filled black squares, endogenous full-length Sup35 aggregates; filled green circles, Sup35(1-61)-GFP-labelled particles; open green circles, Sup35(1-6)-GFP aggregates derived from E. coli. b, Strain typing. A set of yeast centromere-based plasmids encoding the wild type and single mutations of the Sup35 protein (labelled WT, G58D (that is, Gly to Asp mutation at amino acid 58), G44R, S17R and Q15R) as well as yeast 2μ-based multi-copy plasmids encoding the wild-type Sup35(1-6)-GFP or the G20D mutation⁷were introduced into the fused cell by mating with testers which carry the plasmids. The strain type (labelled on the left) is determined by the distinctive pattern of colour responses to the full-length Sup35 proteins. For [VH] the colour responses are (from lane 1 (WT) to lane 5 (Q15R))−(changing little with respect to WT (self reference)),+(a substantial change), ±(an intermediate change), +, +; for [VK] −, −, −, +, −; for [VL] −, −, +, +, +; and for [psi⁻] ,−,−,−,−. Selected cells with Sup35(1-61)-GFP and Sup35(1-61)(G20D)GFP plasmids were observed further by fluorescence microscopy for strain-specific fluorescent labelling⁷. All three [PSI] strains were cured by passage through media containing 4 mM guanidine hydrochloride⁴ (right panel: [PSI⁺], white, pink or dark pink; [psi⁻], red). In this figure, strain-typed colonies were transformed with infectious yeast particles. [VH] particles were used for the top two rows. [VK] particles were used for the third and fourth rows. The fifth to eighth rows were from a [VL] preparation containing particles of both [VK] and [VL] types. The bottom row is a buffer control.

FIG. 2 shows [PSI] amyloid fibres. a, Electron micrographs of the three [PSI] strain protein aggregates. The top row shows negatively stained Sup35(1-61)-GFP-labelled yeast particles. The middle row shows immunoelectron micrographs of the yeast particles. Particles decorated by a mouse anti-GFP monoclonal antibody were probed with a gold-conjugated anti-mouse IgG antibody. The bottom row shows electron micrographs of negatively stained (left) and ice-embedded (right) Sup35(1-61)-GFP amyloid fibres whose growth was nucleated by the yeast particles. At this resolution, fewer curved fibres were observed in [VH] samples than in [VK] and [VL] samples. The morphology of these negatively stained fibres is similar to that of recombinant Ure2(1-65)-GFP fibres²⁶. b, Electron diffraction patterns of ice-embedded unoriented fibre specimens: [VH] (top half), [VK] (bottom-left) and [VL] (bottom-right), showing the characteristic 4.7 Å spacing of the cross-beta structure of amyloids. The 2.3 Å ring is from evaporated gold, used for calibration. The diffuse ring at approximately 3.6 Å spacing is from vitreous ice. The size of the central inelastic scattering maximum depends on the contrast used to display the sharp 4.7 Å ring. A similar X-ray diffraction pattern was reported from spontaneously formed Sup35 fibres from E. coli²⁷. c-e, Evolution of fibre morphologies in a typical seeding cycle described in Table 3. The fluorescence micrographs show yeast-derived [VK] particles (c) and growth after 48 h incubation with E.-coli-derived Sup35(1-61)-GFP (e). The long fibres are sheared by sonication to release numerous smaller seeds (d). f, Electron micrograph of elongated fibres at a magnification similar to e.

FIG. 3 shows the composition of His5-Sup35(1-61)-GFP-Strep(II) samples analysed by SDS-polyacrylamide gel electrophoresis (SDS-PAGE). Sedimented fibres derived from seeding experiments (Table 5b, 5th propagation), and. spontaneously aggregated samples sedimented from Buffer E (Sample I) and Buffer B (Sample II) were analysed by 15% SDS-PAGE (Coomassie Blue staining). All aggregates have fibrous morphology as judged by electron microscopy. SDSdissociated fibres derived from [VH], [VK], and [VL] reactions as well as from Sample I and Sample II (labelled on the top) exhibit identical gel mobilities as the original soluble His5-Sup35(1-61)-GFP-Strep(II) fusion protein. The marker sizes (in Daltons) are indicated on the left.

FIG. 4 shows the morphology of protein aggregates spontaneously formed in His5-Sup35(1-61)-GFP-Strep(II) solutions. Electron microscopy reveals the fibrous morphology of the protein aggregates obtained in Buffer E at 4° C. (left) and in Buffer B at 22° C. (right, both negatively stained). The diameters of the fibres are 173.6±11 Å (Buffer E) and 167±14 Å A (Buffer B). At the current resolution, they are indistinguishable from each other and from fibres seeded by yeast particles (FIG. 4a). Their uniform diameter is similar to that of recombinant Ure2(1-65)-GFP fibres (Baxa, U. et al. Architecture of Ure2p prion filaments J. Biol. Chem. 278, 43717-43727 (2003)).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular fragment of the Sup35 protein is disclosed and discussed and a number of modifications that can be made to a number of molecules including the amino acids are discussed, specifically contemplated is each and every combination and permutation of Sup35 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In particular, disclosed is a peptide comprising amino acids 1-61 of SEQ ID NO: 2 (SEQ ID NO: 11). It was found that this peptide is a prion-determining fragment of the full length Sup35, and that this peptide can form infectious fibrous aggregates (Example 1.) Also disclosed are fusion constructs of this fragment, such as Sup35(1-61)-GFP and Sup35(1-61)-GFP-Strep (II). Fusion proteins are powerful tools in cell biology and function by serving as molecular reporters as well as allowing for protein purification. Methods of making fusion proteins are known in the art.

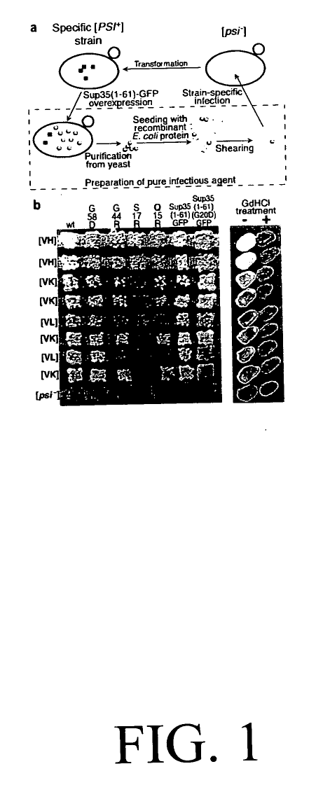

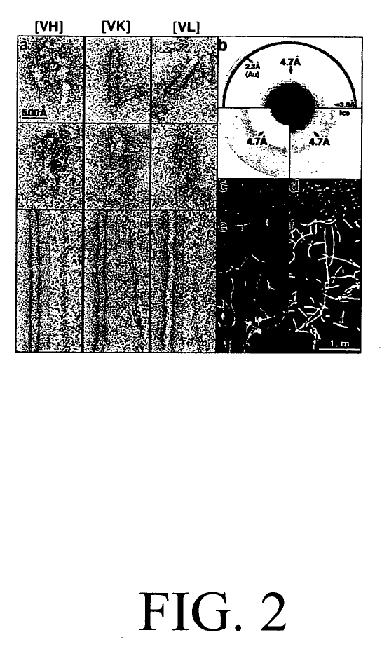

Also disclosed are three different strains of Sup35. These strains are VL, VK, and VH. These strains are able to propagate with strain specificity in prion aggregates (Example 1). Also disclosed are methods of determining prion strain types using the methods disclosed in FIGS. 1 and 2.

1. Homology/Identity

It is understood that one way to define any known variants and derivatives or those that might arise, of the disclosed genes and proteins herein is through defining the variants and derivatives in terms of homology to specific known sequences. For example SEQ ID NO: 1 sets forth the nucleic acid encoding Sup35 and SEQ ID NO: 2 sets forth a particular sequence of the protein encoded by SEQ ID NO: 1, i.e., Sup35. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Also disclosed are fragments of SEQ ID NO: 2. For example, amino acids 1-10, 1-20, 1-30, 1-40, 1-50, 1-60, 1-61, 1-70, 1-80, 1-90, and 1-100, 1-110, 1-120, 1-130, 1-140, 1-150, 1-160, 1-170, 1-180, 1-190, 1-200, 1-210, 1-130, 1-240 (and all points in between) of SEQ ID NO: 2. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Nucleic Acids

There are a variety of molecules disclosed herein that are nucleic acid based, including for example the nucleic acids that encode, for example, Sup35 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, Sup35 as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. For example, the sequences of SEQ ID NOS: 1 and 2 can be found associated with accession numbers AY028659 and AAK26190, respectively. These fall-length sequences are known in the art (Liu et al., PNAS 99:16446-16453, 2002; Ter-Avanesyan et al., Genetics 137:671-676, 1991, both herein incorporated by reference in their entireties, specifically for their teaching regarding the Sup35 protein.) Those of skill in the art understand how to resolve sequence discrepancies and differences and to adjust the compositions and methods relating to a particular sequence to other related sequences. Primers and/or probes can be designed for any sequence given the information disclosed herein and known in the art.

3. Peptides

a) Protein Variants

As discussed herein there are numerous variants of the Sup35 protein and the various strains thereof (VL, VH, and VK) that are herein contemplated. In addition to the functional strain variants there are derivatives of the Sup35 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | AlaA |
| allosoleucine | AIle |
| Arginine | ArgR |
| Asparagines | AsnN |
| aspartic acid | AspD |
| Cysteine | CysC |
| glutamic acid | GluE |
| Glutamine | GlnK |
| Glycine | GlyG |
| Histidine | HisH |
| Isolelucine | IleI |
| Leucine | LeuL |
| Lysine | LysK |
| phenylalanine | PheF |
| Praline | ProP |
| pyroglutamic acid | Glu |
| Serine | SerS |
| Threonine | ThrT |
| Tyrosine | TyrY |
| Tryptophan | TrpW |
| Valine | ValV |

TABLE 2
Amino Acid Substitutions
Original Residue Exemplary Conservative Substitutions,
others are known in the art.

Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 2 sets forth a particular sequence of the Sup35 protein. Specifically disclosed are variants of these and other proteins herein disclosed which have at least, 70% or 75% or 80% or 85% or 90% or 95% homology to the stated sequence, or comprise a fragment of the sequence, such as amino acids 1-61. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1. It is understood that while no amino acid sequence indicates what particular DNA sequence encodes that protein within an organism, where particular variants of a disclosed protein are disclosed herein, the known nucleic acid sequence that encodes that protein in the particular sequence from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Tbba, Biotechnology & Genetic Enginerring Reviews 13:197-216 (1995), Cahill et al., TIBS, 14(10):400-403 (1989); Benner, TIB Tech, 12:158-163 (1994); Tbba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH=CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

b) Markers

The peptide sequences disclosed herein, for example SEQ ID NO: 2, can have a marker product associated therewith. This marker product is used to determine if the peptide has been delivered to the cell and to determine its location therein. A preferred marker is the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to-arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

4. Vectors, Cells, and Methods of Using

Also provided is a vector, comprising a nucleic acid of the present invention, such as SEQ ID NO: 1 or a fragment thereof, such as one encoding amino acids 1-61. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification. The vector can comprise the nucleic acid in pET15b, pSRα-Neo, pPICZα, or pPIC9K. The vector can also comprise a nucleic acid that aids in protein purification, such as Strep(II).

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, such as SEQ ID NO: 1 which encodes SEQ ID NO: 2, wherein the nucleic acid can be expressed by a yeast cell. More specifically, the nucleic acid can be expressed by Pichia pastoris or S. cerevisiae. There are several advantages to yeast expression systems, which include, for example, Saccharomyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFα-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals.

Alternatively, the nucleic acid coding sequences can be fused to a second protein coding sequence, such as Sj26, Strep(II) or beta-galactosidase, used to facilitate purification of the fusion protein by affinity chromatography. The nucleic acid coding sequence can also be fused to a fluorescent protein coding sequence, such as GFP, RFP, YFP, and BFP, for example. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well known methods, which vary depending on the type of cellular host. For example, calcium chloride transformation is commonly utilized for prokaryotic cells, whereas calcium phosphate, DEAE dextran, or lipofectin mediated transfection or electroporation may be used for other eukaryotic cellular hosts.

Alternative vectors for the expression of genes or nucleic acids in mammalian cells, those similar to those developed for the expression of human gamma interferon, tissue plasminogen activator, clotting Factor VIII, hepatitis B virus surface antigen, protease Nexinl, and eosinophil major basic protein, can be employed. Further, the vector can include CMV promoter sequences and a polyadenylation signal available for expression of inserted nucleic acids in mammalian cells (such as COS 7).

Insect cells, for example, can also permit the expression of various proteins. Recombinant proteins produced in insect cells with baculovirus vectors undergo post translational modifications similar to that of wild type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a myeloma cell, a Pichia cell, or an insect cell. The coding sequence for any of the polypeptides described herein can be introduced into an insect cell line, for example, using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines have been developed and include myeloma cell lines, fibroblast cell lines, and a variety of tumor cell lines such as melanoma cell lines. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

The present invention provides a method of making any of the polypeptides or fragments thereof of the present invention, comprising: culturing a host cell comprising a vector that encodes a polypeptide or fragment thereof; and purifying the polypeptide or fragment thereof produced by the host cell. As mentioned above, these polypeptides include, but are not limited to, a polypeptide comprising SEQ ID NO: 2 or fragments thereof, such as amino acid fragment 1-61. The polypeptides of the present invention can also be made by culturing a host cell comprising a nucleic acid that encodes the full-length Sup35 protein (SEQ ID NO:2), purifying the full-length Sup35 protein produced by the host cell and digesting the full-length Sup35 protein with the appropriate enzymes to produce a polypeptide comprising a fragment of SEQ ID NO:2, such as amino acids 1-61, for example.

5. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with Sup35 such that the protein is inhibited from interacting with other Sup35 proteins, thereby creating prions. The antibodies can be tested for their desired activity using the in vitro assays described herein, or by analogous methods, after which their in vivo therapeutic and/or prophylactic activities are tested according to known clinical testing methods.

Also disclosed are antibodies useful in diagnostic methods. For example, disclosed are antibodies specific for SEQ ID NO: 2 (Sup35) that can be used in methods for detecting the presence of [PSI⁺] cells. Specifically, disclosed are antibodies specific for Sup35 (1-61) and fragments thereof. Also disclosed are antibodies specific for the strains disclosed herein such as VH, VK, and VL (Example 1).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Administration of Antibodies

Administration of the antibodies can be done as disclosed herein. Nucleic acid approaches for antibody delivery also exist. The broadly neutralizing anti Sup35 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

6. Pharmaceutical Carriers/Delivery of Pharamceutical Products

As described above, the compositions, such as the antibodies and peptides disclosed herein, can be administered in vivo in a pharmaceutically acceptable carrier for diagnostic methods or methods of treatment. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing prion-associated diseases, the efficacy of the therapeutic Antibody can be assessed in various ways well known to the skilled practitioner. For instance, one of ordinary skill in the art will understand that a composition, such as an antibody, disclosed herein is efficacious in treating or inhibiting any prion-related diseases in a subject by observing that the composition reduces existing prion load or prevents a further increase in prion load. (Prion loads can be measured by methods that are known in the art, for example, detecting amyloid-like plaques).

Compositions that inhibit Sup35 interactions may be administered prophylactically to patients or subjects who are at risk for prion-related diseases. In subjects who have been newly exposed to prions but who have not yet displayed the presence of the related disease in blood or other body fluid, efficacious treatment with an antibody partially or completely inhibits the appearance of the prion-related disease.

Other molecules that interact with Sup35 to inhibit prion interactions which do not have a specific pharmaceutical function, but which may be used for tracking changes within cellular chromosomes or for the delivery of diagnositc tools for example can be delivered in ways similar to those described for the pharmaceutical products.

The disclosed compositions and methods can also be used for example as tools to isolate and test new drug candidates for a variety of prion related diseases.

7. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

8. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

9. Compositions Identified by Screening with Disclosed Compositions/Combinatorial chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed in SEQ ID NO: 2 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, Sup35, are also disclosed. Thus, the products produced using the combinatorial or screening approaches that involve the disclosed compositions, such as, Sup35, are also considered herein disclosed.

It is understood that the disclosed methods for identifying molecules that inhibit the interactions between, for example, prions and other pre-prion peptides, can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. No. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice is attached to a DNA binding domain of a transcriptional activation protein, such as Gal 4, for example. By performing the Two-hybrid technique on this type of system, molecules that bind the extracellular portion of Sup35 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, various prion strains, are also disclosed. Thus, the products produced using the molecular modeling approaches that involve the disclosed compositions, such as the various prion strains, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO: 2 and fragments thereof, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant Ga. (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp.257-267 (1992)). 3. Process for Making the Compositions

Disclosed are processes for making the compositions as well as making the intermediates leading to the compositions. For example, disclosed is a nucleic acid in SEQ ID NO: 1. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO: 1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO: 1 and a sequence controlling the expression of the nucleic acid.

Disclosed are nucleic acid molecules produced by the process comprising linking in an operative way a nucleic acid molecule comprising a sequence encoding a peptide having 80% identity to a peptide set forth in SEQ ID NO: 2 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO: 2, wherein any change from the sequence are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### D. METHODS OF USING THE COMPOSITIONS

1. Methods of Using the Compositions as Research Tools

The disclosed compositions can be used in a variety of ways as research tools. For example, the disclosed compositions, such as SEQ ID NOs: 1 and 2, can be used to study the interactions between Sup35 forming molecules, by for example causing the formation of prions or prion aggregates.

The disclosed compositions can also be used diagnostic tools related to prion-related diseases. For example, the nucleic acid of Sup35 or a fragment thereof can be used as probes to diagnose the presence of Sup35. A peptide suspected of being a prion or a prion-susceptible protein can be confirmed to be such by administering the peptide to a cell, tissue, or organism, and testing for the formation of prion aggregates.

Furthermore, various prion strains, such as VH, VK and VL (Example 1) can be characterized by their response to a panel of Sup35 mutants. For example, yeast centromere-based plasmids encoding the wild type and single mutations of Sup35 protein can be introduced into a fused cell. The strain type can then be determined by distinctive pattern of colour response to full length Sup35 proteins present in the cell.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, Sup35, particularly allelic analysis as it relates to prions and their functions. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C or is at ambient temperature, and pressure is at or near atmospheric.

### 1. Example 1

### Protein-Only Transmission of Three Yeast Prion Strains

Key questions regarding the molecular nature of prions are how different prion strains can be propagated by the same protein and whether they are only protein¹⁻³. The protein-only nature of prion strains has been herein demonstrated in a yeast model, the [PSI] genetic element, which enhances the read-through of nonsense mutations in the yeast Saccharomyces cerevisiae^{4,5}. Infectious fibrous aggregates containing a Sup35 prion-determining amino-terminal fragment labelled with green fluorescent protein were purified from yeast harbouring distinctive prion strains. Using the infectious aggregates as ‘seeds’, elongated fibres were generated in vitro from the bacterially expressed labelled prion protein. De novo generation of strain-specific [PSI] infectivity was demonstrated by introducing sheared fibres into uninfected yeast hosts. The cross-sectional morphology of the elongated fibres generated in vitro was indistinguishable from that of the short yeast seeds, as visualized by electron microscopy. Electron diffraction from the long fibres showed the 4.7 Å spacing characteristic of the cross-beta structure of amyloids. The fact that the amyloid fibres nucleated in vitro propagate the strain-specific infectivity of the yeast seeds implies that the heritable information of distinct prion strains must be encoded by different, self-propagating cross-beta folding patterns of the same prion protein.

The cellular propagation of [PSI] requires an N-terminal prion-inducing fragment of the Sup35 protein⁶. Overexpression of a fusion construct, Sup35(1-6)-GFP, consisting of green fluorescent protein (GFP) fused to the carboxy-terminal of the first 61 amino-acid residues of Sup35, labels numerous small, fast-moving fluorescent particles in essentially every cell harbouring the [PSI] element ([PSI⁺] cell), but produces diffused cytoplasmic labelling in [psi⁻] cells⁷.

The question was whether the labelled particles observed in [PSI⁺] cells could transmit strain-specific phenotypes. To facilitate the purification of [PSI] particles, a C-terminal 10-amino-acid affinity tag, Strep-tag (II), was attached to the fusion protein. Sup35(1-61)-GFP-Strep(II)-labelled particles were isolated from yeast harbouring three different [PSI] strains, designated [VH], [VK] and [VL], and further purified by StrepTactin affinity chromatography⁸. The prion strains could be distinguished by their characteristic responses to a panel of Sup35 mutants⁷. Four of the twelve Sup35 mutants tested in ref. 7 were used for strain typing (FIG. 1b).

An efficient cell fusion method was designed to introduce [PSI] particles into yeast. Two populations of [psi⁻] spheroplasts with complementary genetic markers were mixed with purified particles and induced to fuse by polyethylene glycol treatment. The [PSI] status and strain type of the fused cells, which were selected by the presence of both genetic markers, were then determined (FIG. 1b). As detailed in Table 5, more than 10% of the complementary fused cells were strain-specifically transformed by [VH] (30H/246; that is, 30 [VH] and 216 [psi⁻] out of 246 randomly selected colonies) and [VK] (76K/174) particles; however, in [VL] transformants, all three [PSI] strains were observed (3H, 11K, 52L/248). Overexpression of Sup35(1-61)-GFP-Strep(II) in yeast did not affect the faithful propagation of [VH] and [VK], but resulted in the appearance of [VH], [VK] and [psi⁻] colonies in overnight [VL] cultures with frequencies of approximately 0.2%, 10% and 20%, respectively. A control plasmid without the fusion construct did not affect the propagation of [VL]. The occurrence of [VH] and [VK] strains in [VL] transformants can therefore be traced to the instability of original [VL] cultures when the truncated Sup35 protein was overexpressed. Strain competition experiments, similar to those carried out with the mammalian prion⁹, demonstrated that [VH] particles can dominate [VK] and [VL] particles; whereas [VK] particles can dominate [VL] particles.

[PSI⁺] transformants were not obtained when either similarly obtained preparations from [psi⁻] cells or soluble Sup35(1-61)-GFP (purified from [psi⁻] cells) was used. Sup35(1-61)-GFP-0 particles lacking the Strep(II) affinity tag were also isolated from yeast, and fractioned the preparations by StrepTactin chromatography. Although [PSI] infectivity remained in the flow-through fractions, the infectivity in the column eluates was diminished. These results established that the observed [PSI] infectivity was associated with the Sup35(1-61)-GFP labelled particles, rather than with other fortuitously co-purified factors.

Enzymatic modification experiments further confirmed the proteinaceous nature of the infectious particles. Whereas extended incubation of the [PSI] particles with protease K resulted in complete loss of infectivity, strain-specific propagation of infectivity was unaffected by RNase A treatment (Table 7).

De novo generation of strain-specific infectivity was demonstrated in a set of key experiments where the infectious yeast [PSI] particles were used to nucleate the assembly of a bacterially derived Sup35(1-61)-GFP construct. Yeast [PSI] particles were diluted 40-fold in whole-cell extracts of Escherichia coli, which were engineered to overexpress His₅-Sup35(1-61)-GFP-Strep(II) (that is, Sup35(1-61)-GFP-Strep(II) with an N-terminal affinity tag containing five histidine residues to facilitate purification of the protein on a Ni—NTA column). The reactions were incubated at room temperature in an undisturbed state for 24 h and then briefly sonicated. One-half of the content in the reactions was added to an equal volume of the E. coli extract and incubated for another 24 h. The identical sonication, dilution and incubation were performed once more and the aggregates were collected by ultracentrifugation. The resulting aggregates contained about 160-fold dilution of the original infectious material. The aggregates were resuspended and sonicated in the same buffer as the original seeding particles and were used to transform yeast. Although the newly nucleated aggregates retained the strain-specific infectivity, no activity of the original seeding material, diluted 160-fold and then sonicated, could be detected (Table 1).

Propagation of [PSI] activity requires the recombinant Sup35(1-61) fragment. This was demonstrated by parallel control experiments where the Sup35(1-61) fragment in the E. coli cell extract was replaced with a prion-inducing fragment (amino-acid residues 1-81) of the Ure2 protein, which is essential for the propagation of the yeast prion [URE3]^{5,10}. No [PSI] activity was detected from these reactions (Table 1). As the molecular content of the Ure2(1-81)-containing cell extract mimicked the Sup35(1-61) extract except for the prion-inducing fragments, it is evident that the Sup35(1-61) fragment was necessary for the in vitro propagation of [PSI] infectivity.

It was then determined whether Sup35(1-61)-GFP alone was sufficient for the propagation of [PSI] infectivity. Soluble His₅-Sup35(1-61)-GFP-Strep(II) was purified from the E. coli extract by Ni—NTA affinity chromatography, and was used for the same serial seeding experiments as described above. Although the infectivity of the original seeds was already below detection at 100-fold dilution, the strain-specific infectivity of seeded material nearly always increased after each dilution-propagation cycle (Table 3).

TABLE 3
Propagation of [PSI] activity in vitro

| | Sup35(1-61)-GFP | | Ure(1-81)-GFP | Buffer E | Buffer E | |
|---|---|---|---|---|---|---|
| Prion strain | 1/160 dilution | | 1/160 dilution | 1/160 dilution | 1/80 dilution | |
| [VH] seeds | 39H | (248) | 0 (248) | 0 (248) | 0 | (248) |
| [VK] seeds | 12K | (248) | 0 (235) | 0 (192) | 1K | (248) |
| [VL] seeds | 4L | (248) | 0 (248) | 0 (248) | 0 | (248) |
| Buffer (mock) | 0 | (248) | 0 (231) | 0 (124) | 0 | (248) |

Propagation of [PSI] activity using purified His₅-Sup35(1-61)-GFP-Strep(II)

| | 2nd propagation | | 4th propagation | | 5th propagation | | Buffer E |
|---|---|---|---|---|---|---|---|
| Prion strain | 1/100 dilution | | 1/400 dilution | | 1/800 dilution | | 1/100 dilution |
| [VH] seeds | 1H | (124) | 11H, 1K | (124) | 23H | (124) | 0 (124) |
| [VK] seeds | 12K | (124) | 11K | (124) | 17K | (124) | 0 (124) |
| [VL] seeds | 0 | (124) | 2L | (124) | 3L, 3K | (124) | 0 (124) |
| Buffer (mock) | 0 | (124) | 2K | (124) | 1K | (124) | 0 (124) |

Numbers and strain types of transformed yeast (H, [VH]; K, [VK]; L, [VL]) are listed. Numbers of randomly screened colonies are in parentheses. [PSI] seeds purified from yeast in buffer E or in buffer E alone (first column) were added to extract of E. coli expressing His₅-Sup35(1-61)-GFP-Strep(II) or His₅-Ure2(1-81 GFP-Strep(II) (top) or to purified His₅-Sup35(1-61)-GFP-Strep(II) (bottom). The dilution factor with respect to the original seed in each sample is indicated. Seeds directly diluted in buffer E were used as controls. The same batch of spheroplasts was used for experiments described in the same row. Transformation by mock-seeded samples (bottom row) probably resulted from de novo generation of [VK] in the purified protein solution. Spontaneous aggregation of the recombinant protein, after extended incubation, to form infectious amyloid aggregates was reproducible.

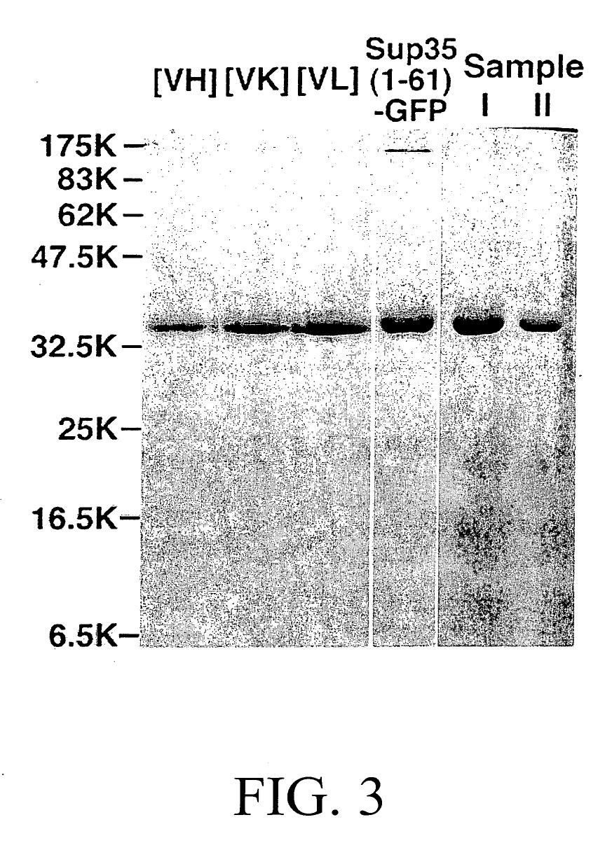

The newly assembled aggregates of different [PSI] strains were analysed by SDS-polyacrylamide gel electrophoresis (PAGE) (FIG. 3) and isoelectric focusing (pH range 3-10, data not shown). The dissociated aggregates exhibited identical gel mobilities to that of the original soluble His₅-Sup35(1-61)GFP-Strep(II) fusion protein. No nucleic acid in purified His₅-Sup35(1-61)-GFP-Strep(II) were detected by gel electrophoresis (ethidium bromide staining; see Example 3), neither did the presence of RNase A in the seeding reactions influence the propagation of strain-specific infectivity (Table 7b). Taken together, these data indicated that the in vitro replication of [PSI] strains only requires the Sup35(1-61)-GFP construct.

The amyloid-like nature of [PSI] strains was next determined. Numerous rod-shaped particles with variable lengths (20-150 nm) and a uniform width of about 170-180 Å were seen by negative staining electron microscopy in the infectious yeast preparations (FIG. 2a, top row). These structures were the only entities recognized by a GFP-specific antibody (FIG. 2a, middle row). As the infectivity was associated with Sup35(1-61)-GFP in yeast preparations (see above), these rods must be the [PSI] determinant. When incubated with purified His₅-Sup35(1-61)-GFP protein, the short rods grew to lengths greater than 1 μm (FIG. 2a, bottom row, and panels e, f). As visualized by negative staining and cryoelectron microscopy, the fibre morphology of the long rods was indistinguishable from that of the short infectious yeast particles used for nucleation. The measured diameters of negatively stained long fibres are 170±11 Å for [VH], 177±9 Å for [VK] and 177±11 Å for [VL]. Electron diffraction from the ice-embedded long fibres showed the 4.7 Å spacing characteristic of the cross-beta structure (FIG. 2b).

The long fibres could be broken into numerous short fibrils by sonication as monitored by fluorescence microscopy (FIG. 2d, e) and electron microscopy (not shown). The multiplication of fibrils thus provided an explanation for the observed increase of infectivity in the serial seeding experiments described in Table 1. This surmise was then tested by comparing the infectivity of seeded aggregates with and without sonication. Yeast-derived pre-sonicated aggregates were used to seed the assembly of E.-coli-derived highly purified His₅-Sup35(1-61)-GFP-Strep(II) at room temperature without agitation. After 48 h, each reaction was divided into two equal portions. One was subjected to brief sonication, while the other left undisturbed. These samples were then used to transform yeast. As controls, the same amount of the yeast aggregates was added in exactly the same way to the same batch of unpolymerized E. coli protein without incubation, and then treated with or without sonication before assaying for infectivity. For every [PSI] strain, brief sonication increases the efficiency of yeast transformation for the incubated, seeded samples compared with the controls (Table 4).

TABLE 4
Increase of [PSI] infectivity by sonic disruption

| | +Incubation | | +Incubation | | −Incubation | | −Incubation | |
|---|---|---|---|---|---|---|---|---|
| Prion strain | +sonication | | −sonication | | +sonication | | −sonication | |
| [VH] seeds | 41H | (248) | 17H | (248) | 0 | (248) | 3H | (248) |
| [VK] seeds | 77K | (248) | 14K | (248) | 17K | (248) | 20K | (248) |
| [VL] seeds | 16L, 1K | (248) | 2L | (248) | 2L, 1K | (248) | 1L, 1K | (248) |

| | | | | | | | |
|---|---|---|---|---|---|---|---|
| Buffer (mock) | 0 | (248) | 0 | (248) | ND | 0 | (248) |

Number and strain types of transformed yeast are listed in the same fashion as in Table 1. Pre-sonicated [PSI] seeds in buffer E as well as buffer E control (first column) were added to highly purified recombinant His₅—Sup35(1-61)-GFP-Strep(II) protein, either immediately before yeast transformation (−incubation) or allowing incubation at room temperature for 48 h before transformation (+incubation). Before transformation, the mixtures were divided into two equal portions. One was treated with brief sonication (+sonication), and the other was left untreated (−sonication). The same batch of spheroplasts was used for all experiments. The moderate increase of infectivity after 48 h incubation in untreated [VH] sample (+incubation, −sonication) but not in untreated [VK] or [VL] samples is reproducible with different batches of seeds. This fact seems to indicate that [VH] fibrils are more fragile ND, not determined.

The enhanced infectivity of the fragmented amyloid fibres can be attributed to the increase in the number of particles after sonication, although fragmentation can also facilitate transformation if the shorter filaments are more efficiently assimilated on fusion of the yeast cells. The sheared fibres were on average longer than the original yeast particles as judged by fluorescence (FIG. 2c, d) and electron microscopy, yet they were more numerous and thus collectively more infectious. This point was illustrated further by the serial seeding experiments described in Table 1 where sheared fibres in each growth cycle were of similar sizes, but the infectivity of the samples almost always increased after each cycle. In yeast the propagation of [PSI] depends on Hsp104 activity¹¹. The Hsp104 protein is believed to fragment the Sup35 aggregates in [PSI⁺] cells, thus allowing the replication and propagation of [PSI]¹². This experiment, correlating mechanical shearing of fibres with increase in infectivity in vitro, seems to mirror the proposed in vivo mechanism.

The data presented so far, namely the nucleated growth of strain-specific [PS1] infectivity in solutions of purified recombinant protein, strongly support the protein-only nature of [PSI] strains as well as the adequacy of the Sup35(1-61) construct in propagating strain-specific biological activity in vitro (FIG. 1a). Although catalytic amounts of yeast particles were used as seeds to encode strain-type information, their presence was successively reduced by serial seeding in the experiments presented in Table 1. To corroborate further the ‘protein-only’ assertion, an independent set of transformation experiments were carried out, similar to those of Sparrer et al.¹³, using bacterially derived Sup35(1-61)-GFP fibres, which were spontaneously aggregated from pure protein solutions.

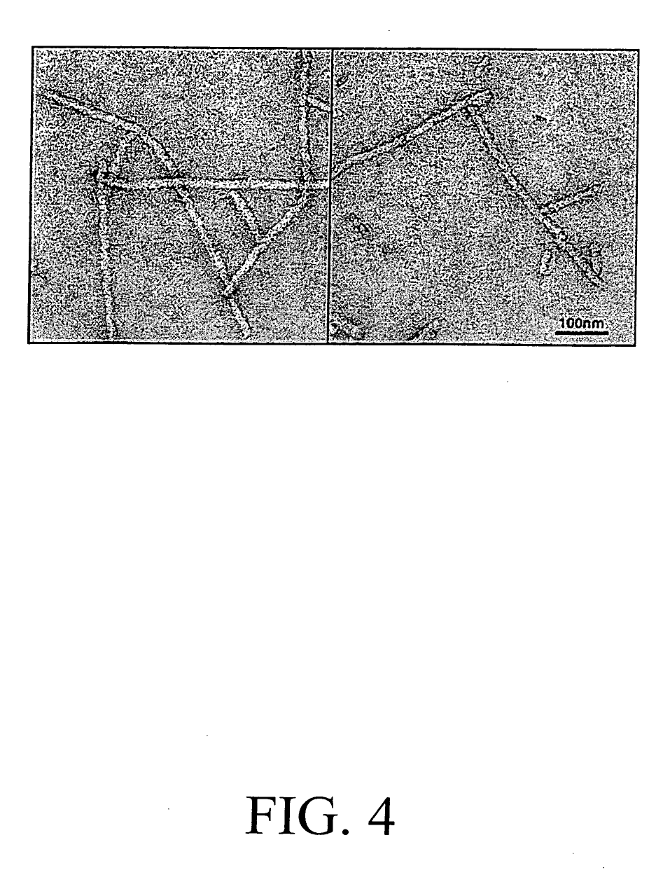

Two different aggregates were prepared without seeding by extended incubation of E.-coli-derived His₅-Sup35(1-61)-GFP-Strep(II). Sample I, obtained from buffer E (100 mM TrisHCl, 1 mM EDTA, 2.5 mM desthiobiotin, pH 8.0) at 4° C., was concentrated by centrifugation and resuspension in the same buffer. Sample II was obtained from buffer B (20 mM TrisHCl, 100 mM NaCl, 500 mM Imidazole, pH 7.6) at 22° C., collected by centrifugation, and resuspended in buffer E. Electron microscopy revealed amyloid fibres with similar morphology in both samples (FIG. 4). The samples were checked by SDS-PAGE (FIG. 3) and isoelectric focusing. The dissociated samples appeared identical with regard to their monomer size and the electric charge compared to those of the soluble His₅-Sup35(1-61)-GFP-Strep(II). Yeast colonies transformed by sample I exhibited the [VH] strain phenotype (38H/248, 20H/248 for 5/8 dilution) and colonies transformed by sample II displayed the [VK] phenotype (2K/124, 2K/248, same sample; the same spheroplast preparations were used for the latter and the 5/8 dilution of sample I). The observed [PSI] induction cannot merely be attributed to the fact that overexpression of the Sup35 protein induces [PSI] de novo in yeast⁴⁻⁵—such a procedure lacks prion-strain specificity¹⁶. Instead, the strain-specific transformation indicates the transmission of specific genetic information from the two samples, thus qualifying spontaneously aggregated Sup35 amyloid fibres as bona fide infectious agents. As a further control, the lack of de novo [PSI] generation was confirmed in the experimental yeast genetic background when His₅-Sup35(1-61)-GFP was overexpressed from a multi-copy yeast plasmid (Example 3). These observations demonstrate that [PSI] infectivity can also arise from self-assembly of the pure E. coli protein construct, which was never in contact with yeast cell extract.

The results demonstrate that [PSI] strains are propagated by infectious amyloid aggregates of the Sup35 prion-forming domain, thus resolving current ambiguity regarding the molecular nature of [PSI]^{13,16,17} and the mechanism for its strain variations¹⁸. ‘Protein-only’ [PSI] implies that the prion strain differences—distinguished by testing their responses with a panel of mutant Sup35 proteins—must be structural. Although the in vivo propagation of [PSI] after infection probably involves complex cellular interactions¹⁹, the data clearly show that the first 61 amino-acid residues of the Sup35 protein are sufficient for encoding the strain-specific infectivity. The challenge now is to characterize the structural differences in the cross-beta folding patterns of the same 61-amino-acid fragment that encodes the heritable information for different prion strains.

### 2. Example 2

### General Methods

Plasmids and Yeast Genetic Backgrounds

The construction of YEp-CUP1-Sup35(1-61)-GFP-Strep(II)-T and YEp-CUP1-His₅-Sup35(1-61)-GFP-Strep(II)-T is described in Example 3. The plasmids YCp-I-SUPFs (URA3) have been described previously⁷.

The E. coli plasmids pHIS-Sup35(1-61)-GFP-Strep(II) and pHIS-Ure(1-81)-GFP-Strep(II) were constructed on the T7 expression vector pMW172 (ref. 20). Yeast strains were derived from 5V-H19 (MATa SUQ5 ade2-1(UAA) can1-100 leu2-3,112 ura3-52)⁶. For [PSI] transformation, g-αh-5V-H19 (MATαSUQ5 ade2-1(UAA) can1-100 leu2-3,112 ura3-52 [psi⁻] [pin⁻]) and g-αh-5V-H19 ΔHIS4 [YCplac111] (g-αh-5V-H19 his4::loxP-kanMX-loxP [YCplac111 (LEU2)]) were used. [PSI⁺] [PIN⁺] derivatives were used for strain competition. Escherichia coli strain BLR (DE3)/pLysS (ref. 21) was purchased from Novagen.

Purification of [PSI] Particles

[PSI] particles were purified from cell lysates of 5V-H19 (MATa/MATa [PIN⁺] derivatives) transformed with YEp-CUP1-Sup35(1-61)-GFP-Strep(II)-T (Example 3). Lysates were placed on top of 30% (g ml⁻¹) sucrose (in buffer A (20 mM TrisHCl, 100 mM NaCl, 5 mM imidazole, pH 7.6)) for 2 h sedimentation at 200,000 g. Aggregates were re-suspended in 2 ml buffer A plus 50 μg avidin, sonicated briefly, and then loaded to 1 ml StrepTactin columns (IBA). The columns were washed at room temperature with 10 vol. of buffer W (100 mM TrisHCl, 1 mM EDTA, pH 8.0), 15 vol. of buffer A, and then eluted by 6×0.5 ml buffer E (buffer W plus 2.5 mM desthiobiotin). The third and fourth elution fractions contained most of the [PSI] particles and were collected. The first 1 ml wash was used as unbound fractions for transformation experiments (Supplementary Table S1). Soluble Sup35(1-61)-GFP-Strep(II) was similarly purified from a [psi⁻] [pin⁻] derivative, except that the supernatant above the sucrose cushion was used for subsequent StrepTactin chromatography.

Escherichia coli Protein Preparation and in Vitro Seeding

Bacterial protein overexpression was induced by 1 mM isopropyl-β-D-thiogalactopyranoside at an optical density of 0.6 at 595 nm (LB media, 37° C.) in BLR (DE3)/pLysS cells transformed with pHIS-Sup35(1-61)-GFP-Strep(II) or pHIS-Ure(1-81)-GFP-Strep(II). Escherichia coli cell extracts were prepared 3 h after induction by removing debris (12,000 g sedimentation, twice) from cell lysates, which were obtained by sonication of 0.5 g ml⁻¹ cell suspension in buffer A plus 1 mM phenylmethylsulphonyl fluoride.

Cell extracts were loaded onto 7 ml Ni—NTA affinity columns (Qiagen) at 4° C. The columns were washed extensively with buffer A. Protein was eluted by buffer B (buffer A plus 500 mM imidazole) and stored immediately at −80° C. Samples thus prepared were used for seeding experiments as purified protein. For experiments in Table 2, a sample was further purified by StrepTactin affinity chromatography and used immediately. Typical recombinant protein concentrations for seeding experiments were between 10 and 40 μM. In this report ‘ultracentrifugation’ refers to 80,000 g sedimentation on top of 1 ml 30% (g ml⁻¹) sucrose cushion for 2 h and ‘brief sonication’ indicates a 10 s sonic disruption with 6 W power output. Unpolymerized protein solutions were checked by fluorescence microscopy for lack of visible aggregation before seeding experiments.

Yeast Transformation by [PSI] Particles

Yeast spheroplasts were prepared from g-αh-5V-H19 and g-αh-5V-H19 ΔHIS4 [YCplac111] in buffer Z1 (1.2 M sorbitol, 10 mM TrisHCl (pH 7.5), 30 mM CaCl₂). Fifty microlitres of each spheroplast suspension were mixed with 8 μl solution containing prion particles, incubated at 22° C. for 15 min, and then combined. One millilitre of buffer Z2 (20% (w/v) polyethylene glycol 3350, 10 mM TrisHCl (pH 7.5), 30 mM CaCl₂) was added to the mixture. After 15 min incubation at 22° C., spheroplasts were collected and allowed to recover in 150 μl Z3 medium (1 M sorbitol, 30 mM CaCl₂, one-third strength YPAD²², 7 mg l⁻¹ uracil, 7 mg l⁻¹ histidine, 33 mg l⁻¹ leucine) at 30° C. for 30 min. Top agar (SC-LEU, HIS, with 1.2 M sorbitol, 8 ml) was then added to the spheroplasts to plate on top of SC-LEU, HIS agar²² containing 1.2 M sorbitol. Karyogamy in the 5V-H19 background was efficient^{23,24}. A control transformation by pure sample buffer was included for every set of experiments. A transformed colony was never obtained from these controls.

Strain Typing

Randomly selected colonies were transferred from top agar to SC-LEU, HIS plates and incubated at 30° C. for 5 days to allow colony colour to fully develop. All white, pink and dark pink colonies were tested for [PSI] by mating on SC-URA, LEU, HIS plates with a set of MATa/MATa [psi⁻] testers, transformed with YEp-CUP1-N1-GFP-T, YEp-CUP1-N1(G20D)-T, YCp-I-Sup35F, YCp-I-Sup35F(G58D), YCp-I-Sup35F(G44R), YCp-I-Sup35F(S17R), and YCp-I-Sup35F(Q15R). The strain types were determined by characteristic colour responses⁷ (FIG. 1b). See Example 3 for more details.

### 3. Example 3

### Supplementary Methods

Plasmids Construction

The yeast plasmid YEp-CUP1-N1-GFP-T, expressing Sup35(1-61)-GFP by a copper inducible CUP1 promoter, was described. The plasmid YEp-CUP1-Sup35(1-61)-GFPStrep(II)-T was constructed by replacing the Sph I/Not I fragment from YEp-CUP1-N1-GFP-T with a PCR (polymerase chain reaction) fragment amplified by primers CYK-72(5′-GGGAGGGCATGCCTTCAACGA TTTCTATGATGC-3′) (SEQ ID NO: 3) and CYK-139 (5′-GATTGCGGCCGCTTACTATTTTTCGAACTGCGGGTGGCTCCAGG CAGACTTGTACAGCTCGTCCATGC-3′) (SEQ ID NO: 4) on the YEp-CUP1-N1-GFP-T template. To obtain the plasmid YEP-CUP1-H5-Sup35(1-61)-GFP-Strep(II)-T, a DNA fragment encoding Met-Gly-Ser2-His5-Ser2-Gly was derived by pairing oligonucleotides CYK-153 (5′-GATCCATGGGCAGCAGCCATCATCACC ACCATAGCAGCGGCG-3′) (SEQ ID NO: 5) and CYK-154(5′-GATCCGCCGC TGCTATGGTGGTGATGATGGC TGCTGC CCATG-3′) (SEQ ID NO: 6) and inserted into the BamH I site of YEp-CUP1-Sup35(1-61)-GFP-Strep(II)-T. Plasmids YCp-I-SUPF(LEU)'s were constructed by inserting the Sph I/EcoR I fragments from respective YCp-I-SUPF's into the plasmid YCplac111 (Gietz, R. D. & Sugino, A. New yeast-Escherichia coli shuttle vectors constructed with in vitro mutagenized yeast genes lacking six-base pair restriction sites. Gene 74, 527-534 (1988)). The E. coli expression plasmid pHISSup35(1-61)-GFP-Strep(II) was obtained by inserting the BamH I/EcoR I fragment of YEp-CUP1-Sup35(1-61)-GFP-Strep(II)-T into the T7 expression vector pMW172 (Way, M., Pope, B., Gooch, J., Hawkins, M. & Weeds, A. G. Identification of a region in segment 1 of gelsolin critical for actin binding. EMBO J. 9, 4103-4109 (1990)), followed by insertion of an Nde I/BamH I fragment obtained by hybridizing oligonucleotides CYK-8 (5′-TATGGGCA GCAGCCATCATCATCACCATAG CAGCGGCG) (SEQ ID NO: 7) and CYK-9, (5′-GATCCGCCGCTGCTATGG TGATGATGATGGC TGCTGCCCA) (SEQ ID NO: 8). The pHIS-Ure(1-81)-GFP-Strep(II) plasmid was derived by replacing the BamH I/Xma I fragment of pHISSup35(1-61)-GFP-Strep(II) with a PCR fragment obtained by primers CYK-3 (5′-TTGTAGG ATCCATGATGAATAACAACGGCAACCAAGTG) (SEQ ID NO: 9) and CYK-144 (5′-CTAGCCCGGGGTAAGGTATTCTTGATATTATTCTC) (SEQ ID NO: 10) on yeast genomic DNA template. All PCR-derived DNA inserts were sequenced.

[PSI] Curing

[PSI+] colonies were grown in 2 ml YPAD4 containing 4 mM guanidine hydrochloride at 30° C. for 24 hours. Aliquots of the cultures were then spread on YPD(Sherman, F. Getting started with yeast. Methods Enzymol. 194, 3-21 (1991)) plates to isolate [psi−] colonies.

Preparation of Yeast Cell Lysates

MATa/MATa, [PIN+] derivatives of 5V-H19 harbouring three [PSI] prion strains were transformed with YEp-CUP1-Sup35(1-61)-GFP-Strep(II)-T and grown in 2 litres of synthetic dextrose minimal medium (SD medium), supplemented with adenine and leucine. CuSO4 was added to 4 mM final concentration at mid-log phase to induce Sup(1-61)-GFP-Strep(II) expression for 12 hours at 30° C. Freshly harvested cells were resuspended in 10 ml Y-PER Yeast Protein Extraction Reagent (Pierce, Ill.) containing 1 mM phenylmethylsulfonyl fluoride (PMSF) and incubated at 30° C. for an hour. Disrupted cells were collected, re-suspended in 10 ml Y-PER plus 10 ml buffer A and 1 mM PMSF, and homogenized by agitation with an equal volume of glass beads (0.5mm diameter). Cell debris was removed by centrifugation twice at 12,000×g for 30 minutes each.

Preparation of Yeast Spheroplasts

Colonies of g-αh-5V-H19 and g-αh-5V-H19 ΔHIS4 [YCplac111] were grown at 30° C. overnight in 10 ml synthetic complete medium (SC) and 10 ml SC medium lacking leucine (SC-LEU), respectively. Overnight cells were collected by centrifugation and allowed to grow in 10 ml YPAD rich media for 90 minutes at 30° C. Cells were harvested, washed and re-suspended in 1 ml 1.2M sorbitol and treated with 100 units of Lyticase (Sigma, L-5263) until more than 90% of the cells were converted to spheroplasts, which were then washed and re-suspended in 400 ml Buffer Z1 (1.2 M sorbitol, 10 mM TrisHCl (pH 7.5), 30 mM CaCl2).

More on Strain Typing

The strain typing methods were independent of cell ploidyl, which was not controlled in the cell fusion protocol. For strain typing yeast expressing Sup35(1-61)-GFP-Strep(II), aliquots of yeast cultures were spread on SC-URA plates 12 hours post protein induction. Proportional numbers of white, pink, and dark pink colonies were then randomly selected from the plates to mate (on SC-URA, LEU) with MAT α/MAT α[psi−] testers, which are transformed with YCp-I-Sup35F(LEU)'s. While the presence of the expression plasmid influenced the background nonsense suppression efficiencies, it did not alter the strain specific relative colour responses. The exact relation between our strain-typing system and another widely used “strong-weak” strain-typing system (Derkatch, I. L., Chernoff, Y. O., Kushnirov, V. V., Inge-Vechtomov, S. G. & Liebman, S. W. Genesis and variability of [PSI] prion factors in Saccharomyces cerevisiae. Genetics 144, 1375-1386 (1996)) remains to be established. In particular, it remains to be clarified whether the [VH], [VK], or [VL] strain assigned here can have “strong-weak” sub-variations.

Detection of Nucleic Acids by Gel Electrophoresis

Solutions containing about 500 μg of purified E. coli His5-Sup35(1-61)-GFP were mixed with phenol/chloroform/isoamyl alcohol (25:24;1,v/v). Potential nucleic acids were collected from the aqueous phase by ethanol precipitation and subjected to agarose gel electrophoresis (1.8% (w/v) in TBE (89 mM TrisHCl, 89 mM boric acid, 2 mM EDTA (pH8.0)), ethidium bromide staining).

Lack of de novo [PSI] Generation in g-αh-5V-H19/g-αh-5V-H19 ΔHIS4 [YCplac111]

To reaffirm the lack of de novo [PSI] generation in our experimental setting where [pin−] cells were used ([PSI] can be efficiently induced by overexpression of Sup35p in a [PIN+] cell but much less so in a [pin−] cell (Derkatch, I. L., Bradley, M. E., Zhou, P., Chemoff, Y. O. & Liebman, S. W. Genetic and environmental factors affecting the de novo appearance of the [PSI+] prion in Saccharomycescerevisiae. Genetics 147, 507-519 (1997)), four [psi−] colonies obtained from spheroplast fusion were randomly selected, and then transformed with a plasmid overexpressing His5-Sup35(1-61)-GFP. To avoid selection bias, fused [psi−] cells were taken from a control plate where spheroplasts were transformed with a buffer only. Plasmid-transformed colonies (one each) were randomly selected, grown in media inductive to protein overexpression, and then plated. No [PSI] induction was observed out of more than 1000 colonies from each plate. As a positive control, it was observed that the same plasmid could induce [PSI] in about 1% of [PIN+] cells with otherwise identical genetic background with roughly equal frequency for [VH] and [VK] strains—no [VL] strain was observed (King, C.-Y. Supporting the structural basis of prion strains: induction and identification of [PSI] variants. J. Mol. Biol. 307, 1247-1260 (2001)).

### F. REFERENCES

1. Prusiner, S. B. Prions. Proc. Natl Acad. Sci. USA 95, 13363-13383 (1998).
2. Dickinson, A. G. & Outram, G. W. Genetic aspects of unconventional virus infections: the basis of the virino hypothesis. Ciba Found. Symp. 135, 63-83 (1988).
3. Weissmann, C. A ‘unified theory’ of prion propagation. Nature 352, 679-683 (1991).
4. Cox, B. S., Tuite, M. F. & McLaughlin, C. S. The psi factor of yeast: a problem in inheritance. Yeast 4, 159-178 (1988).
5. Wickner, R. B. [URE3] as an altered URE2 protein: evidence for a prion analog in Saccharomyces cerevisiae. Science 264, 566-569 (1994).
6. Ter-Avanesyan, M. D., Dagkesamanskaya, A. R., Kushnirov, V. V. & Smimov, V. N. The SUP35 omnipotent suppressor gene is involved in the maintenance of the non-Mendelian determinant [PSI⁺] in the yeast Saccharomyces cerevisiae. Genetics 137, 671-676 (1994).
7. King, C.-Y. Supporting the structural basis of prion strains: induction and identification of [PSI] variants. J. Mol. Biol. 307, 1247-1260 (2001).
8. Skerra, A. & Schmidt, T. G. Use of the Strep-Tag and streptavidin for 10 detection and purification of recombinant proteins. Methods Enzymol. 326, 271-304 (2000).
9. Dickinson, A. G. et al. Extraneural competition between different scrapie agents leading to loss of infectivity. Nature 253, 556 (1975).
10. Maddelein, M. L. & Wickner, R. B. Two prion-inducing regions of Ure2p are nonoverlapping. Mol. Cell. Biol. 19, 4516-4524 (1999).
11. Chernoff, Y. O., Lindquist, S. L., Ono, B., Inge-Vechtomov, S. G. & Liebman, S. W. Role of the chaperone protein HsplO4 in propagation of the yeast prion-like factor[PSI+]. Science 268, 880-884 (1995).
12. Kushnirov, V. V. & Ter-Avanesyan, M. D. Structure and replication of yeast prions. Cell 94, 13-16 (1998).
13. Sparrer, H. E., Santoso, A., Szoka, F. C. & Weissman, J. S. Evidence for the prion hypothesis: induction of the yeast [PSI⁺] factor by in vitro-converted Sup35 protein. Science 289, 595-599 (2000).
14. Chernoff, Y. O., Derkach, I. L. & Inge-Vechtomov, S. G. Multicopy SUP35 gene induces de-novo appearance of psi-like factors in the yeast Saccharomyces cerevisiae. Curr. Genet. 24, 268-270 (1993).
15. Derkatch, I. L., Chernoff, Y. O., Kushnirov, V. V., Inge-Vechtomov, S. G. & Liebman, S. W. Genesis and variability of [PSI] prion factors in Saccharomyces cerevisiae. Genetics 144, 1375-1386 (1996).
16. Liebman, S. W. Progress toward an ultimate proof of the prion hypothesis. Proc. Natl Acad. Sci. USA 99, 9098-9100 (2002).
17. Tuite, M. F. & Cox, B. S. Propagation of yeast prions. Nature Rev. Mol. Cell. Biol. 4, 878-890 (2003).
18. Uptain, S. M., Sawicki, G. J., Caughey, B. & Lindquist, S. L. Strains of [PSI⁺] are distinguished by their efficiencies of prion-mediated conformational conversion. EMBO J. 20, 6236-6245 (2001).
19. Liu, J.-J., Sondheimer, N. & Lindquist, S. L. Changes in the middle region of Sup35 profoundly alter the nature of epigenetic inheritance for the yeast prion [PSI+]. Proc. Natl Acad. Sci. USA 99, 16446-16453 (2002).
20. Way, M., Pope, B., Gooch, J., Hawkins, M. & Weeds, A. G. Identification of a region in segment 1 of gelsolin critical for actin binding. EMBO J. 9, 4103-4109 (1990).
21. Studier, F. W., Rosenberg, A. H., Dunn, J. J. & Dubendorff, J. W. Use of T7 RNA polymerase to direct expression of cloned genes. Methods Enzymol. 185, 60-89 (1990).
22. Sherman, F. Getting started with yeast. Methods Enzymol. 194, 3-21 (1991).
23. Harashima, S., Takagi, A. & Oshima, Y. Transformation of protoplasted yeast cells is directly associated with cell fusion. Mol. Cell. Biol. 4, 771-778 (1984).
24. Rose, M. D., Price, B. R. & Fink, G. R. Saccharomyces cerevisiae nuclear fusion requires prior activation by alpha factor. Mol. Cell. Biol. 6, 3490-3497 (1986).
25. Wischik, C. et al. Structural characterization of the core of the paired helical filament of Alzheimer disease. Proc. Natl Acad. Sci. USA 85, 4884-4888 (1988).
26. Baxa, U. et al. Architecture of Ure2p prion filaments. J. Biol. Chem. 278, 43717-43727 (2003).
27. Serio, T. R. et al. Nucleated conformational conversion and the replication of conformational information by a prion determinant. Science 289, 1317-1321 (2000).
28. King, C.-Y. Supporting the structural basis of prion strains: induction and identification of [PSI] variants. J. Mol. Biol. 307, 1247-1260 (2001).
29. Gietz, R. D. & Sugino, A. New yeast-Escherichia coli shuttle vectors constructed with in vitromutagenized yeast genes lacking six-base pair restriction sites. Gene 74, 527-534 (1988).
30. Way, M., Pope, B., Gooch, J., Hawkins, M. & Weeds, A. G. Identification of a region in segment 1 of gelsolin critical for actin binding. EMBO J. 9, 4103-4109 (1990).
31. Sherman, F. Getting started with yeast. Methods Enzymol. 194, 3-21 (1991).
32. Derkatch, I. L., Chemoff, Y. O., Kushnirov, V. V., Inge-Vechtomov, S. G. & Liebman, S. W.Genesis and variability of [PSI] prion factors in Saccharomyces cerevisiae. Genetics 144,1375-1386 (1996).

33. Derkatch, I. L., Bradley, M. E., Zhou, P., Chemoff, Y. O. & Liebman, S. W. Genetic and environmental factors affecting the de novo appearance of the [PSI+] prion in Saccharomycescerevisiae. Genetics 147, 507-519 (1997).

TABLE 5
Strain-specific transformation by [PSI] particles.

| | | [VH] particles | | [VK] particles | | [VL] particles | |
|---|---|---|---|---|---|---|---|
| | | +Tag | −Tag | +Tag | −Tag | +Tag | −Tag |
| Exp | Unbound | 7H(243) | 16H(146) | | | 0H | 1H |
| I | Fraction | | | 46K(126) | 97K(134) | 0K | 1K |
| | | | | | | 1L(248) | 13L(242) |
| | Bound | 30H(246) | 2H(248) | | | 3H | |
| | Fraction | | | 76K(174) | 0(206) | 11K | |
| | | | | | | 52L(248) | 0(241) |
| Exp | Unbound | 13H(124) | 33H(124) | | | 0H | 0H |
| II | Fraction | | | 20K(124) | 24K(124) | 0K | 0K |
| | | | | | | 4L(124) | 7L(124) |
| | Bound | 41H(124) | 0(124) | | | 0H | |
| | Fraction | | | 28K(124) | 0(124) | 2K | |
| | | | | | | 4L(124) | 0(124) |

Sup35(1-61)-GFP associated particles were isolated from yeast harbouring three [PSI] strains [VH], [VK], and [VL] (labelled on the top). They were purified by StrepTactin affinity chromatography and used to transform yeast devoid of [PSI]. Numbers of transformed colonies and their strain-types are indicated in the table (H: [VH]; K: [VK]; L: [VL]; the total number of colonies screened is parenthesized). [PSI] activity was associated with the Sup35(1-61)-GFP fusion construct as demonstrated by differential partitions of infectivity between the column flow-through (unbound fraction) and the elute fraction (bound fraction), when GFP fusion constructs with or without the Strep(II) affinity tag (labelled “+Tag”, “−Tag”, respectively) were expressed. The same batch of spheroplasts was used for all experiments in Exp. II and each column in Exp. I. Independently prepared [PSI] particles of the same prion strain were used in Exp. I and II.

TABLE 6
Competition among [PSI⁻] strains

| | [VH] | [VK] | [VL] | Buffer |
|---|---|---|---|---|
| | particles | particles | particles | (control) |
| αh-5V-H19 [VH] | 124H (124) | 248H (248) | 248H (248) | 124H (124) |
| + | | | | |
| αh-5V-H19 [VH] | | | | |
| ΔHIS4 [YCp111] | | | | |
| αh-5V-H19 [VK] | 11H | 0H | N.D. | 0H |
| + | 55K (70) | 127K (128) | | 90K (96) |
| αh-5V-H19 [VK] | 0L | 0L | | 0L |
| ΔHIS4 [YCp111] | 4[psi⁻] | 1[psi⁻] | | 6[psi⁻] |
| αh-5V-H19 [VL] | 24H | 0H | N.D. | 0H |
| + | 0K (122) | 53K (121) | | 0K (119) |
| αh-5V-H19 [VL] | 96L | 65L | | 114L |
| ΔHIS4 [YCp111] | 2[psi⁻] | 3[psi⁻] | | 5[psi⁻] |
| αh-5V-H19 [psi⁻] | N.D. | 0H | 0H | 0H |
| + | | 27K (104) | 1K (116) | 0K (100) |
| αh-5V-H19 [psi⁻] | | 0L | 10L | 0L |
| ΔHIS4 [YCp111] | | 77[psi⁻] | 105[psi⁻] | 100[psi⁻] |

Two populations of complementary [PSI⁺] spheroplasts (the leftmost column) were fused together in the presence of prion particles purified from yeast harbouring a different [PSI] strain (labelled on the top). Numbers and strain types of transformed yeast are listed in the same fashion as Supplementary Table S1. N. D.=not determined. The same batch of spheroplasts was used for experiments described in each row of the table.

TABLE 7
Proteinaceous nature of [PSI].

| | [VH] | [VK] | [VL] | | Buffer |
|---|---|---|---|---|---|
| | particles + | particles + | particles + | | (mock) + |
| a | PK | PK | PK | | PK |
| +incubation | 0 (69) | 0 (60) | 0 | (81) | — |
| −incubation | 3H (78) | 6K (68) | 4L, 3K | (99) | 0 (79) |

| | | |
|---|---|---|
| | +Sup35(1-61) | +Buffer |
| b | +RNase A | −RNase A |

| | | | |
|---|---|---|---|
| [VH] seeds | 19H | (123) | 1H (118) |
| [VK] seeds | 15K | (123) | 3K (122) |
| [VL] seeds | 4L,3K | (124) | 1L (107) |
| Buffer (mock) | 1H | (124) | 0 (124) |

Numbers and strain types of transformed yeast are listed in the same fashion as in Supplementary Table S1. (a) Protease K treatment. Yeast derived prion particles kept at 30° C. were treated with protease K (6.6 μg/ml final concentration) either 24 hours prior to transformation (+incubation) or right before transformation (−incubation). Extended incubation with protease K abolishes [PSI] infectivity. (b) RNase A treatment. Ten microlitres of yeast derived prion particles (left column) were treated with 0.1 mg (7 unit) RNase A at 22° C. for 1 hour before 190 μl recombinant His₅-Sup35(1-61)-GFP-Strep(II) in Buffer B were added to the prion-RNase A mixture. The seeding reactions were allowed to proceed in the presence of RNase A for 48 hours at 22° C. The aggregates were then collected, sonicated, resuspended in Buffer E, and used for transformation experiments (+Sup35(1-61), +RNase A). As controls, identical amount of prion particles without RNase A was diluted in Buffer E directly, sonicated, and used for transformation (+Buffer, −RNAse A). The same batch of spheroplasts was used for all experiments.

1. A composition comprising SEQ ID NO: 11 or a functional fragment thereof.

2. The composition of claim 1, further comprising Green Fluorescent Protein (GFP).

3. The composition of claim 2, further comprising Strep(II).

4. An isolated nucleic acid encoding the amino acid sequence set forth in SEQ ID NO: 11.

5. A vector comprising the nucleic acid of claim 4.

6. A cell containing the vector of claim 5.

7. A method of introducing a prion into a cell, comprising the steps of:
a) diluting yeast [PSI] particles in whole-cell extracts of bacteria, which were engineered to overexpress a polypeptide comprising SEQ ID NO: 11,
b) incubating the yeast particles and whole-cell extracts of bacteria,
c) repeating steps a-b until the desired dilution is achieved, and
d) transforming yeast with the product of step c.

8. The method of claim 7, wherein the bacteria is E. coli.

9. The method of claim 7, wherein the overexpressed polypeptide comprises a histidine tag.

10. The method of claim 7, wherein the overexpressed polypeptide comprises GFP.

11. The method of claim 7, wherein the overexpressed polypeptide comprises Strep(II).


SEQUENCE LISTING (11 sequences)

SEQ ID NO: 1 - DNA, 713 bp, Artificial Sequence
  Description of Artificial Sequence; note = synthetic construct
atgtcggattcaaaccaaggcaacaatcagcaaaactaccagcaatacagccagaacggtaaccaacaacaaggtaacaacagataccaaggttatcaagcttacaatgctcaagcccaacctgcaggtgggtactaccaaaattaccaaggttattctgggtaccaacaaggtggctatcaacagtacaatcccgacgccggttaccagcaacagtataatcctcaaggaggctatcaacagtacaatcctcaaggcggttatcagcagcaattcaatccacaaggtggccgtggaaattacaaaaacttcaactacaataacaatttgcaaggatatcaagctggtttccaaccacagtctcaaggtatgtctttgaacgactttcaaaagcaacaaaagcaggccgctcccaaaccaaagaagactttgaagcttgtctccagttccggtatcaagttggccaatgctaccaagaaggttgacacaaaacctgccgaatctgataagaaagaggaagagaagtctgctgaaaccaaagaaccaactaaagagccaacaaaggtcgaagaaccagttaaaaaggaggagaaaccagtccagactgaagaaaagacggaggaaaaatcggaacttccaaaggtagaagaccttaaaatctctgaatcaacacataataccaacaatgccaatgttaccagtgctgatgcctt

SEQ ID NO: 2 - protein, 237 aa, Artificial Sequence
  Description of Artificial Sequence; note = synthetic construct
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVDTKPAESDKKEEEKSAETKEPTKEPTKVEEPVKKEEKPVQTEEKTEEKSELPKVEDLKISESTHNTNNANVTSADA

SEQ ID NO: 3 - DNA, 33 bp, Artificial Sequence
  Description of Artificial Sequence; note = synthetic construct
gggagggcatgccttcaacgatttctatgatgc

SEQ ID NO: 4 - DNA, 68 bp, Artificial Sequence
  Description of Artificial Sequence; note = synthetic construct
gattgcggccgcttactatttttcgaactgcgggtggctccaggcagacttgtacagctcgtccatgc

SEQ ID NO: 5 - DNA, 42 bp, Artificial Sequence
  Description of Artificial Sequence; note = synthetic construct
gatccatgggcagcagccatcatcaccaccatagcagcggcg

SEQ ID NO: 6 - DNA, 42 bp, Artificial Sequence
  Description of Artificial Sequence; note = synthetic construct
gatccgccgctgctatggtggtgatgatggctgctgcccatg

SEQ ID NO: 7 - DNA, 38 bp, Artificial Sequence
  Description of Artificial Sequence; note = synthetic construct
tatgggcagcagccatcatcatcaccatagcagcggcg

SEQ ID NO: 8 - DNA, 40 bp, Artificial Sequence
  Description of Artificial Sequence; note = synthetic construct
gatccgccgctgctatggtgatgatgatggctgctgccca

SEQ ID NO: 9 - DNA, 38 bp, Artificial Sequence
  Description of Artificial Sequence; note = synthetic construct
ttgtaggatccatgatgaataacaacggcaaccaagtg

SEQ ID NO: 10 - DNA, 35 bp, Artificial Sequence
  Description of Artificial Sequence; note = synthetic construct
ctagcccggggtaaggtattcttgatattattctc

SEQ ID NO: 11 - protein, 61 aa, Artificial Sequence
  Description of Artificial Sequence; note = synthetic construct
MSDSNQGNNQQNYQQYSQNGNQQQGNNRYQGYQAYNAQAQPAGGYYQNYQGYSGYQQGGYQ